(19) United States
(12) Patent Application Publication
(10) Pub. No.: US 2006/0189855 A1
(43) Pub. Date: Aug. 24, 2006

(54) Device for detecting a sleeping state judging device, method of detecting a sleeping state judging method, and computer program product

(75) Inventors: Akihisa Moriya, Kanagawa (JP); Takuji Suzuki, Kanagawa (JP); Kazushige Ouchi, Kanagawa (JP); Kenichi Kameyama, Kanagawa (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Kabushiki Kaisha Toshiba, Minato-ku (JP)

(21) Appl. No.: 11/326,511

(22) Filed: Jan. 6, 2006

(30) Foreign Application Priority Data
Jan. 14, 2005 (JP) 2005-008235

(51) Int. Cl. A61B 5/00; A61B 5/02; A61B 5/103

(52) U.S. Cl. 600/301; 600/595; 600/500

(57) ABSTRACT

A device for detecting a sleeping state that judges a sleeping state of a subject includes a brain activity measuring unit that measures an index which reflects brain activities and which indicates an extent of changes in an activity state of a brain of the subject during sleeping at every given unit time; and a sleeping state judging unit that judges the sleeping state at a given time in a time window which covers one cycle of sleeping, based on the index which reflects the brain activities and which is measured by the brain activity measuring unit at every given unit time within the time window.

### CROSS-REFERENCE TO RELATED APPLICATIONS

This application is based upon and claims the benefit of priority from the prior Japanese Patent Application No. 2005-008235, filed on Jan. 14, 2005; the entire contents of which are incorporated herein by reference.

### BACKGROUND OF THE INVENTION

1. Field of the Invention

The present invention relates to a device for detecting a sleeping state, a method of detecting a sleeping state and a computer program product for sleeping state judgment that judge a sleeping state of a subject.

2. Description of the Related Art Conventionally, as a method for specifying a sleeping state, there is known a method of recording a brain wave, myoelectricity and the like using a polysomnogram and judging the sleeping state based on the recorded brain wave, myoelectricity and the like. However, in the method of measuring the brain wave, the myoelectricity and the like, electrodes need to be attached to a body, which increases the burden on a subject.

As a method in which the attachment of the electrodes is not needed, there is known a method of analyzing the fluctuation in cardiac beats based on RR intervals of the pulse wave to judge an autonomic nerve state. Based on sympathetic nerve activities and parasympathetic nerve activities obtained in this technique, the sleeping state is judged (for example, see Japanese Patent Application Laid-Open No. H7-143972). Thereby, the sleeping state at a predetermined time during a sequence of sleeping can be judged.

However, in the method of judging the sleeping state based on the RR intervals of the pulse wave, a sleep cycle of a human, which is said to be 1.5 to 2.0 hours, is not taken into consideration. Furthermore, circadian rhythm and individual differences are not taken into consideration, either. The base of the RR intervals is varied depending on the circadian rhythm. Accordingly, if threshold values utilized in the sleeping state judgment are set uniformly, the sleeping state cannot be judged accurately.

Thus, since the RR intervals of the pulse wave are affected by the circadian rhythm or the like, the sleeping state judgment based on the RR intervals disadvantageously lacks accuracy.

### SUMMARY OF THE INVENTION

According to one aspect of the present invention, a device for detecting a sleeping state that judges a sleeping state of a subject, includes a brain activity measuring unit that measures an index which reflects brain activities and which indicates an extent of changes in an activity state of a brain of the subject during sleeping at every given unit time; and a sleeping state judging unit that judges the sleeping state at a given time in a time window which covers one cycle of sleeping, based on the index which reflects the brain activities and which is measured by the brain activity measuring unit at every given unit time within the time window.

According to another aspect of the present invention, a method of judging a sleeping state of a subject, includes measuring an index which reflects brain activities and which indicates an extent of changes in an activity state of a brain of the subject during sleeping at every given unit time; and judging the sleeping state at a given time in a time window which covers one cycle of sleeping, based on the index which reflects the brain activities and which is measured at every given unit time within the time window, among the measured indexes which reflects the brain activities.

A computer program product according to still another aspect of the present invention causes a computer to perform the method according to the present invention.

In the sleeping state judging device according to the present invention, since the brain activity measuring unit measures an index which reflects brain activities and which indicates the extent of changes in the activity state of the brain of the subject during sleeping at every given unit time, and the sleeping state judging unit judges the sleeping state at the given time in the time window which covers one cycle of sleeping, based on the index which reflects the brain activities and which is measured by the brain activity measuring unit at every unit time within the time window, an influence of an error caused by circadian rhythm or the like can be reduced without an increasing burden on the subject, whereby the more accurate judgment of the sleeping state can be realized.

### BRIEF DESCRIPTION OF THE DRAWINGS

FIG. 1 is a block diagram of an overall configuration of a device for detecting a sleeping state according to a first embodiment;

FIG. 2 is a view of one example of the attachment of a device for detecting a sleeping state;

FIG. 3 is a view of another example of the attachment of the sleeping state judging device;

FIG. 4 is a chart for explaining a manner of calculation of pulse period data by a pulse period data calculating unit;

FIG. 5 is a chart for explaining an acquisition process by an autonomic nerve index data processing unit which acquires autonomic nerve indexes LF and HF from a power spectrum value;

FIG. 6 is a flowchart of a sleeping state judgment process by the sleeping state judging device according to the first embodiment;

FIG. 7 is a detailed flowchart of a body movement data acquisition process described with reference to FIG. 6;

FIG. 8 is a chart for explaining a time window;

FIG. 9 is a chart of an autonomic nerve index obtained during sleeping;

FIG. 10 is a detailed flowchart of the sleeping state judgment process explained in FIG. 6;

FIG. 11 is a detailed flowchart of the sleeping state judgment process by clustering; and

FIG. 12 is a chart showing a time window set by a time window setting unit of the sleeping state judging device according to a second embodiment.

### DETAILED DESCRIPTION OF THE INVENTION

Hereinafter, the embodiments of a device for detecting a sleeping state, a method of detecting a sleeping state and a computer program product according to the present invention are explained in detail with reference to the accompanying drawings. The embodiments do not limit the present invention.

First, a device for detecting a sleeping state according to a first embodiment is described. The sleeping state judging device according to the first embodiment can judge a sleeping state of a subject during sleeping and sound a wake-up alarm during REM sleeping, which results in comfortable wake-up feelings.

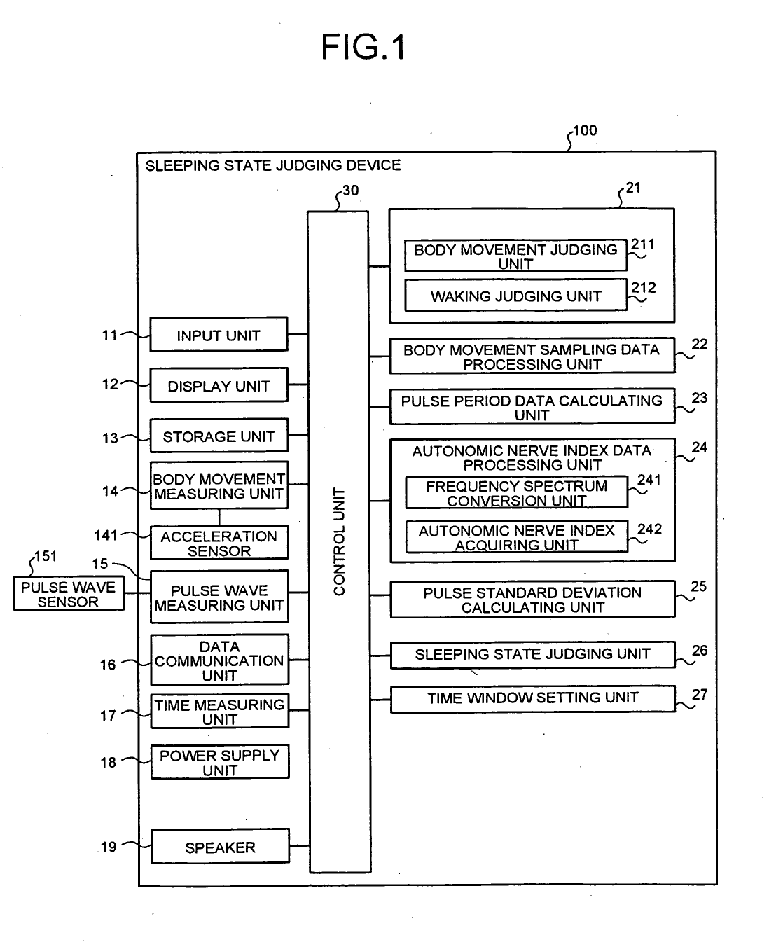

FIG. 1 is a block diagram of an overall configuration of the sleeping state judging device according to the first embodiment. As shown in the figure, the sleeping state judging device 100 has an input unit 11, a display unit 12, a storage unit 13, a body movement measuring unit 14, a pulse wave measuring unit 15, a data communication unit 16, a time measuring unit 17, a power supply unit 18, a speaker 19, a sleeping/waking judging unit 21, a body movement sampling data processing unit 22, a pulse period data calculating unit 23, an autonomic nerve index data processing unit 24, a pulse standard deviation calculating unit 25, a sleeping state judging unit 26, a time window setting unit 27, and a control unit 30.

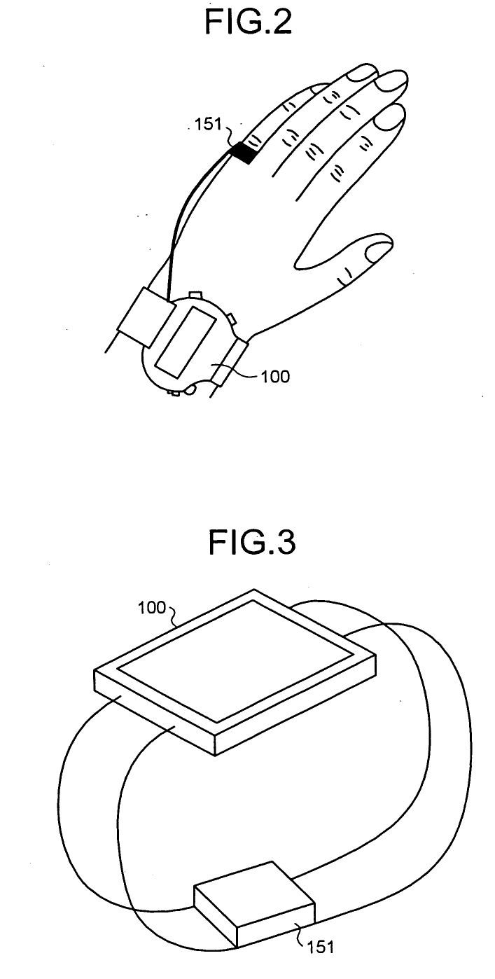

Here, an example of the attachment of the sleeping state judging device 100 as shown in FIG. 1 is explained. FIG. 2 is a view of one example of the attachment of the sleeping state judging device 100 shown in FIG. 1. Furthermore, FIG. 3 is a view of another example of the attachment of the sleeping state judging device 100 shown in FIG. 1. Though in FIG. 2, a pulse wave sensor 151 is attached to a finger, and the body of the sleeping state judging device 100 is attached to a wrist like a wrist watch, the pulse wave sensor 151 can be attached to a palm with an adhesive tape. Alternatively, the pulse wave sensor 151 using infrared radiation or a red light emitting diode (LED) and the body of the sleeping state judging device 100 can be integrated as shown in FIG. 3 and attached onto the artery of the wrist.

Returning to FIG. 1, the input unit 11 is a switch by which a user turns the power on/off, or requests or instructs the switching of the display. The display unit 12 is a display device for displaying a sleeping state judgment result, and specifically, is a liquid crystal display (LCD) or the like.

The storage unit 13 stores measurement data such as pulse wave data and body movement data, data after processing such as pulse period data, and data such as threshold values for judging the sleeping state. The storage unit 13 is, specifically, a flash memory or the like.

The body movement measuring unit 14 measures acceleration data as the body movement data indicating the body movement of the subject and performs data conversion. The body movement measuring unit 14 has an acceleration sensor 141. The acceleration sensor 141 is an accelerometer measuring the acceleration of −2 g to 2 g in triaxial directions, and is mounted inside the body of the sleeping state judging device 100. The body movement measuring unit 14 adjusts a gain and an offset of analog data of the acceleration sensor 141 in an adjustment circuit. The analog data is converted after the adjustment to a digital value by a 10-bit analog-digital (A/D) converter and the resulting digital value is output to the control unit 30.

The pulse wave measuring unit 15 measures the pulse wave data of the subject and performs data conversion. The pulse wave measuring unit 15 has the pulse wave sensor 151. The pulse wave sensor 151 includes a blue LED and a photodiode. A skin surface of a finger is irradiated with light and fluctuations in reflected light caused by the changes in blood flow inside capillary vessels are captured by the photodiode for measurement of the pulse wave. The pulse wave measuring unit 1 further converts an output current from the photodiode of the pulse wave sensor 151 into a voltage by a current-voltage converter. The voltage is amplified by an amplifier and subjected to filtering by a high pass filter (cut-off frequency: 0.1 Hz) and a low pass filter (cut-off frequency: 50 Hz). Then, the processed voltage is converted to a digital value by the 10-bit A/D converter and output to the control unit 30.

The data communication unit 16 is a communication unit performing data communication with a personal computer and a personal digital assistant (PDA) terminal via wireless local area network (LAN) or the like, and specifically, is Bluetooth™ or the like. Furthermore, the time measuring unit 17 is a device for measuring a time, and specifically, is a real-time clock integrated circuit (IC) or the like. Furthermore, the power supply unit 18 is a power source supplying the power of the sleeping state judging device 100, and specifically, is a battery. The speaker 19 has an alarm function to sound an alarm at a desired time

The sleeping/waking judging unit 21 judges whether or not the subject is awake based on the body movement data of the subject. The sleeping/waking judging unit 21 has a body movement judging unit 211 and a waking judging unit 212. The body movement judging unit 211 determines a body movement when the fluctuation amount of the body movement data is larger than a preset threshold value. The waking judging unit 212 determines that the body movement corresponding to the body movement data occurs during waking when an occurrence frequency of the body movement determined by the body movement judging unit 211 is equal to or higher than a threshold value, and determines that the body movement corresponding to the body movement data occurs during sleeping when the occurrence frequency of the body movement is lower than the threshold value.

The body movement sampling data processing unit 22 time-differentiates the acceleration data in the triaxial directions which is obtained from the body movement measuring unit 14 to obtain differential coefficients of the acceleration in the triaxial directions, and further obtains a fluctuation amount of the body movement data which is a square root of a sum of squares of each of the differential coefficients of the acceleration in the triaxial directions and a body movement amount which is an average of the fluctuation amounts of the body movement data in the pulse period. The body movement sampling data processing unit 22 provides the fluctuation amount of the body movement data and the body movement amount to the body movement judging unit 211 as the data for body movement judgment.

The pulse period data calculating unit 23 samples the pulse wave data from the pulse wave of the subject, and processes the relevant pulse wave data to obtain the pulse period data. Here, the pulse period data is time interval data of one cycle of the pulse wave of the subject.

Specifically, the pulse period data calculating unit 23 samples the pulse wave data from the pulse wave. A sequence of sampled pulse wave data is time-differentiated for the removal of a direct current fluctuation component from the sequence of pulse wave data. Further after the removal of the direct current fluctuation component, a maximum value and a minimum value of the pulse wave data approximately one second before and after a processing point in the sequence of pulse wave data are acquired. A value between the maximum value and the minimum value is set a threshold value. For example, with a difference between the maximum value and the minimum value defined as an amplitude, a value of 90% of the amplitude from the minimum value is used as a predetermined threshold value.

Furthermore, times when the value of the sequence of pulse wave data after the removal of the current fluctuation component coincides with the threshold value are calculated, and the pulse wave period data is obtained from the interval between the calculated times.

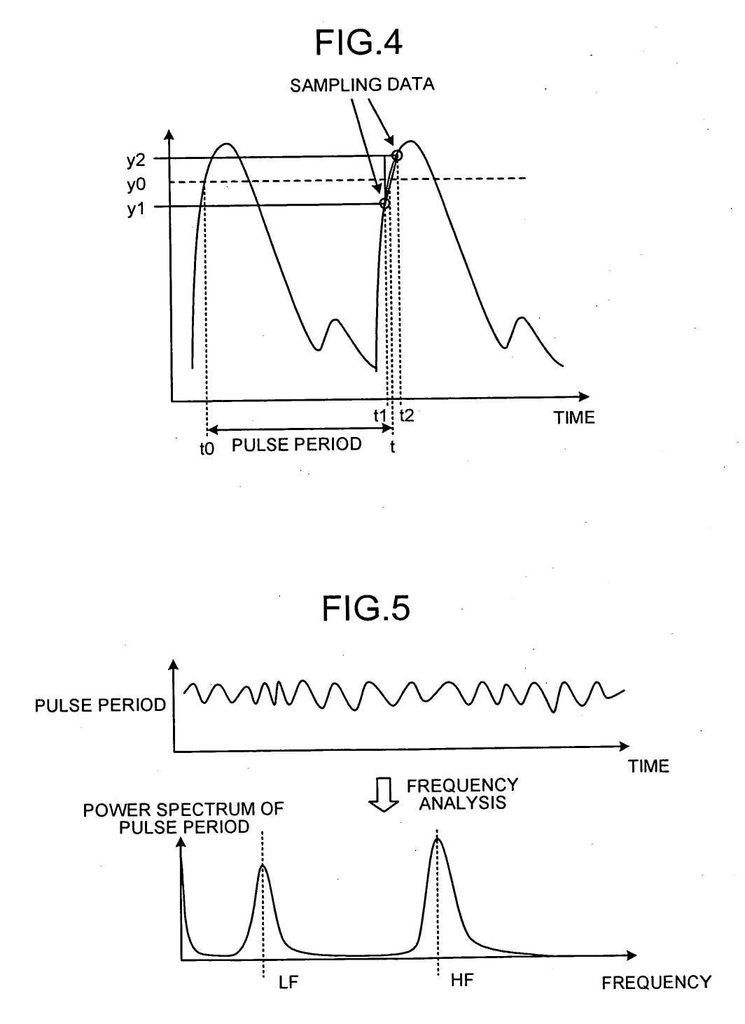

Here, more specifically, a calculation method in which the pulse period data calculating unit 23 shown in FIG. 1 calculates the pulse period data is explained. FIG. 4 is a chart provided for the description of a manner of calculation of the pulse period data by the pulse period data calculating unit 23 shown in FIG. 1.

As shown in FIG. 4, a time corresponding to a threshold value y0 is set as t0. Further, a value of the pulse wave data immediately before exceeding the threshold value y0 is set as y1 and the corresponding time is set as t1. Moreover, a value of the pulse wave data immediately after having exceeded the threshold value y0 is set as y2, and the corresponding time is set as t2. A time t corresponding to a value of the data coinciding with the threshold value is calculated from the formula 1.
t=t1+(t2−t1)×(y0−y1)/(y2−y1) (1)

The pulse period corresponds to a time interval “t−t0” at this time. Accordingly, “t−t0” is calculated as the pulse period data.

Returning to FIG. 1, the autonomic index data processing unit 24 acquires two autonomic nerve indexes, i.e., an index LF in a low-frequency domain (about 0.05 to 0.15 Hz) and an index HF in a high-frequency domain (about 0.15 to 0.4 Hz) for judging the sleeping state.

The autonomic nerve index data processing unit 24 has a frequency spectrum conversion unit 241 and an autonomic nerve index acquiring unit 242. The frequency spectrum conversion unit 241 converts the sequence of pulse period data processed by the pulse period data calculating unit 23 to a frequency spectrum distribution by an analysis technique such as Fast Fourier Transform (FFT) method.

The autonomic nerve index acquiring unit 242 calculates the autonomic nerve indexes LF and HF from a plurality of power spectrum values of the sequence pulse period data converted to the frequency spectrum distribution by the frequency spectrum conversion unit 241. Specifically, an arithmetic average of a total value of three points, namely, peak value of the power spectrum, and points before and after the relevant peak value at even intervals from the relevant peak value is taken as LF or HF.

The process in which the autonomic nerve index data processing unit 24 shown in FIG. 1 calculates the autonomic nerve indexes LF and HF from the values of the power spectrum is explained. FIG. 5 is a chart for explaining the process in which the autonomic nerve index data processing unit 24 shown in FIG. 1 acquires the autonomic nerve indexes LF, HF from the values of the power spectrum. The autonomic nerve index data processing unit 24 performs frequency analysis using FFT method for the pulse period data at even intervals generated by the pulse period data calculating unit 23. Thereby, the sequence of pulse period data is converted to the frequency spectrum distribution.

The upper graph of FIG. 5 shows the pulse period data obtained by the pulse period data calculating unit 23. The lower graph of FIG. 5 shows the spectrum distribution of the power spectrum resulting from the frequency analysis of the pulse period data shown in the upper graph of FIG. 5. As shown in FIG. 5, based on the power spectrum distribution, LF and HF which are the autonomic nerve indexes can be acquired.

As the frequency analysis method, FFT method is preferable in terms of reducing the burden of the data processing. As another example, the AR model, maximum entropy method, wavelet method or the like may be used as the frequency analysis method.

The pulse period data calculating unit 23 and the autonomic nerve index data processing unit 24 according to the first embodiment may function as a brain activity measuring unit. Furthermore, the autonomic nerve index according to the first embodiment may serve as an index which reflects brain activities. In other words, in the first embodiment, the autonomic nerve index is calculated as an index accompanying the changes in the activity state of the brain during sleeping.

Returning to FIG. 1 again, the pulse standard deviation calculating unit 25 calculates a standard deviation of the pulse wave data for one minute. The sleeping state judging unit 26 judges the sleeping state based on the autonomic nerve index obtained by the autonomic nerve index data processing unit 24 and the pulse standard deviation calculated by the pulse standard deviation calculating unit 25. The time window setting unit 27 sets a detection time of the data to be processed by the autonomic nerve index acquiring unit 242 and the like.

The control unit 30 is a control unit that controls the entire sleeping state judging device 100, and controls the processing request to each of the processing units and the flow of the data according to a request and an instruction given by the subject. Specifically, upon receiving a request of the subject, the control unit 30 controls ON/OFF of the power supply, the start of the sleeping state judgment function, the display of the result of sleeping state judgment and the like.

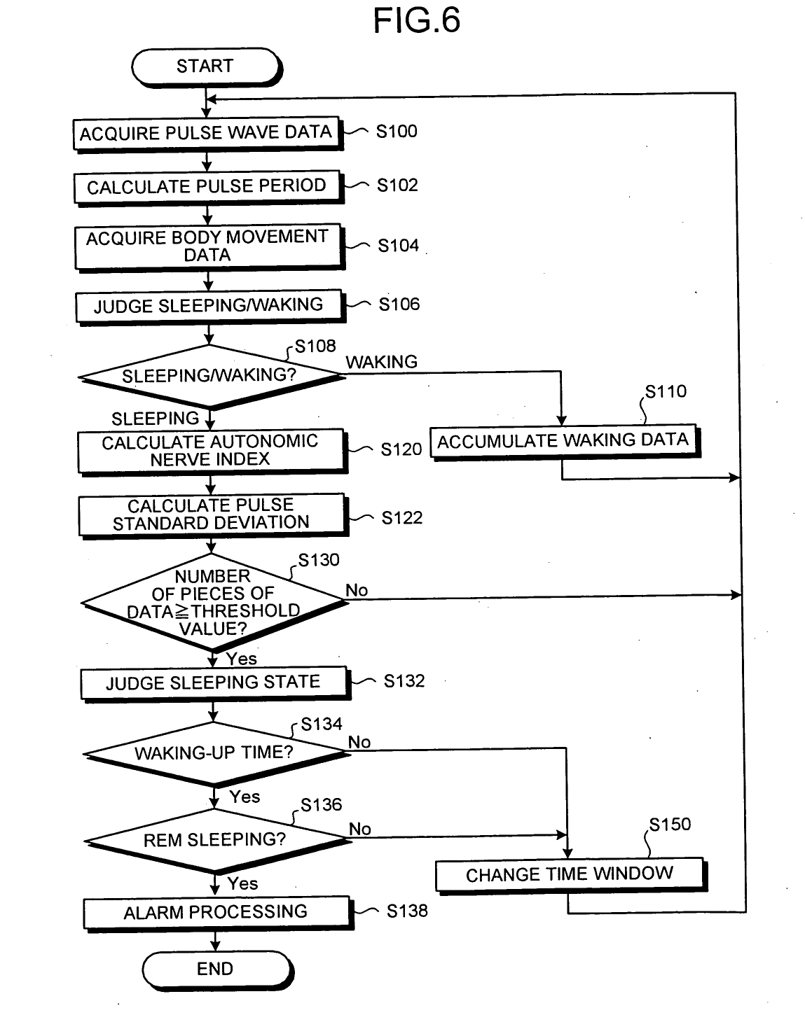

FIG. 6 is a flowchart showing the sleeping state judgment process by the sleeping state judging device 100 according to the first embodiment. First, the subject operates the input unit 11 of the sleeping state judging device 100 to turn the power on and activate the sleeping state judgment function. Further, a desired wake-up time is input. It is desirable that time value in a range of one cycle or more of sleeping, for example, 6 o'clock to 8 o'clock, is input as the wake-up time.

The pulse wave measuring unit 15 of the sleeping state judging device 100 starts the acquisition of the pulse wave data from the pulse wave sensor 151 (step S100). The pulse period data calculating unit 23 calculates the pulse period data, based on the pulse wave data acquired by the pulse wave measuring unit 15 (step S102). At the same time, the body movement measuring unit 14 starts the acquisition of the body movement data (step S104). Specifically, the body movement data is calculated based on the acceleration data detected by the acceleration sensor 141.

In the first embodiment, the pulse wave data is detected every one minute, and the acceleration data is detected every one second. Further, the time window setting unit 27 sets a time window of 100 minutes.

The sleeping/waking judging unit 21 judges whether the subject is in a sleeping state or in a waking state, based on the body movement occurrence frequency calculated by the body movement judging unit 211 (step S106). The method for judging whether the subject is in the sleeping state or in the waking state is explained later.

When the subject is in the waking state (step S108, waking), the waking data indicating that the subject is in the waking state is accumulated in association with a detection time when the relevant waking state is detected (step S110).

When the subject is in the sleeping state (step S108, sleeping), the autonomic nerve index data processing unit 24 calculates the autonomic nerve index (step S120). The calculated autonomic nerve index is accumulated in association with the detection time of the pulse wave data corresponding to the relevant autonomic nerve index. Further, the pulse standard deviation calculating unit 25 calculates a standard deviation of the pulse wave data for one minute (step S122). The calculated pulse standard deviation is accumulated in association with the detection time of the pulse wave data corresponding to the relevant pulse standard deviation.

When the number of accumulated pieces of the above-explained data to be processed is equal to or more than a threshold value (Yes in step S130), the sleeping state judging unit 26, employing a time within the time window set by the time window setting unit 27 as the detection time, judges the sleeping state based on the autonomic nerve index, the pulse standard deviation and the waking data which are accumulated in association with the relevant detection time (step S132). Next, the judged sleeping state is accumulated in association with the corresponding detection time. Further, the display unit 12 may display the result of judgment.

Next, when the current time is the waking-up time and the sleeping state at this time is judged to be REM sleeping (Yes in step S134 and Yes in step S136), the alarm is sounded (step S138).

For example, when the period from 6 o'clock to 8 o'clock in the morning is set as the waking-up time, the alarm can be sounded by the speaker 19 at the detection time of the data judged to be REM sleeping. Then, the sleeping state judgment process by the sleeping state judging device 100 is completed.

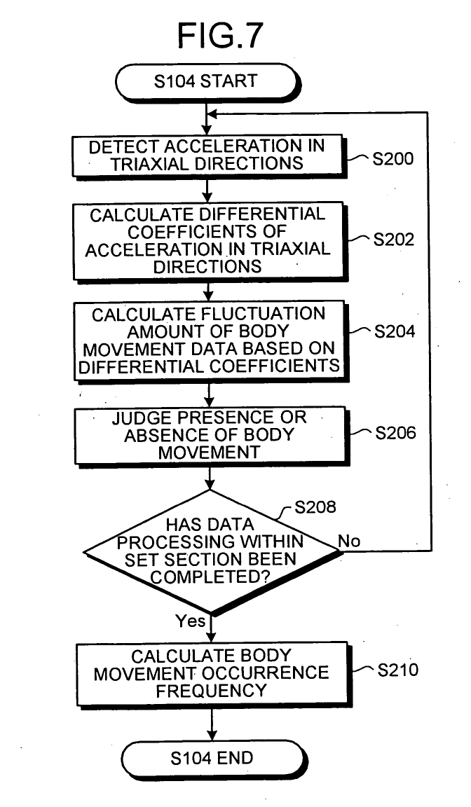

FIG. 7 is a detailed flowchart of the body movement data acquisition process (step S104) described with reference to FIG. 6.

First, the acceleration sensor 141 detects the acceleration in the triaxial directions (step S200). Next, the body movement measuring unit 14 time-differentiates the acceleration data in the triaxial directions based on the acceleration detected by the acceleration sensor 141 to calculate the differential coefficients of the acceleration in the triaxial directions (step S202). Next, the fluctuation amount of the body movement data, which is a square root of a sum of squares of the respective differential coefficients of the acceleration in the triaxial directions is calculated (step S204). Next, the body movement judging unit 211 receives the body movement data from the body movement sampling data processing unit 22 to judge if there is the body movement (step S206).

Specifically, the body movement judging unit 211 judges that there is the body movement when the fluctuation amount of the body movement data is larger than the threshold value. As the threshold value, it is preferable to use 0.01 G, for example, that is a minimum value for the minute body movement used in a body movement meter.

When the process from step S200 to step S206 is completed for the acceleration detected within a set section, which is a desired time interval, (Yes in step S208), the body movement judging unit 211 calculates the body movement occurrence frequency in the set section (step S210). Here, according to the first embodiment, the set section is one minute.

The waking judging unit 212 judges that the body movement occurs while the subject is awake when the occurrence frequency of the body movement determined by the body movement judging unit 211 is equal to the threshold value or more. Further, when the occurrence frequency of the body movement is less than the threshold value, the waking judging unit 212 determines that the body movement occus while the subject is sleeping. It is preferable that the threshold value at this time is 20 times/minute, for example. The threshold value at this time may be decided based on the body movement occurrence frequency at the past waking time.

Next, the process in step S130 and later explained in reference to FIG. 6 is explained in detail. The threshold value of the number of pieces of data used in step S130 is a value corresponding to the time window set by the time window setting unit 27. According to the first embodiment, the pulse wave is detected every minute and the time window is set to 100 minutes. Accordingly, the threshold value of the number of pieces of data in step S130 is 100. Thus, when the pulse wave data for 100 minutes is accumulated, the judgment of the sleeping state is performed based on the accumulated pulse wave data of 100 minutes.

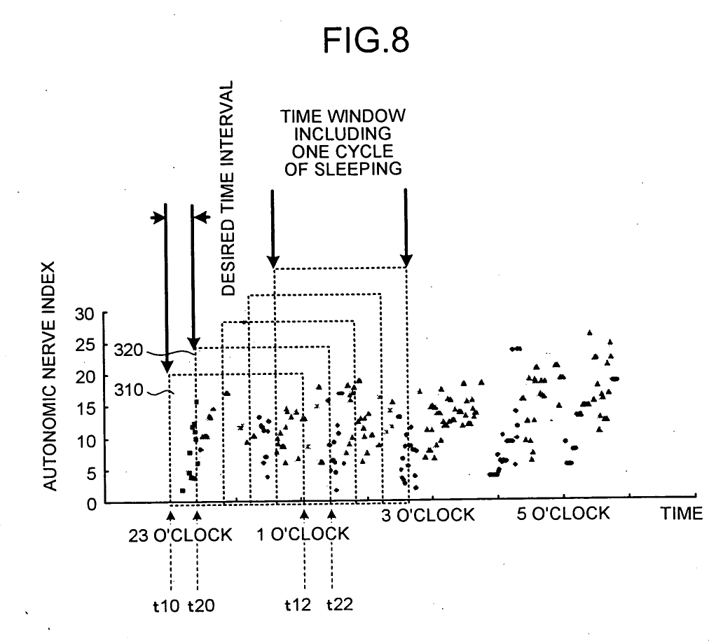

FIG. 8 is a chart for explaining the time window. The time window setting unit 27 according to the first embodiment sets a time interval including one cycle of sleeping as the time window. One cycle of sleeping is 90 minutes. Accordingly, the time window setting unit 27 sets the time interval of 90 minutes or more as the time window. The time window shown in FIG. 8 is 100 minutes.

Based on the autonomic nerve index within a time window 310 in which a time t10 is the start time and a time t12 is the end time, the sleeping state judging unit 26 judges the sleeping state at the time t12 which is the end time of the time window 310 when the time t12 passes. Similarly, based on the autonomic nerve index within a time window 320 in which a time t20 is the start time, the sleeping state judging unit 26 judges the sleeping state at a time t22 which is the end time of the time window 320. In the first embodiment, the time interval for judging the sleeping state is 25 minutes.

Thus, judging the sleeping state every 25 minutes allows the sleeping state to be judged in real time. In terms of valuing real-time judgment, it is preferable that the time interval for judging the sleeping state is shorter than the interval of the time window.

As explained in FIG. 8, in the first embodiment, when the time window is set shorter than the time interval of the time window, the sleeping state at a detection time is judged from the respective processes for a plurality of time windows. In this case, there is a possibility that respective judgment results are different. However, in terms of valuing the real-time judgment, it is preferable that the sleeping state judged from the process for the time window including a later time in the sleeping states judged from the respective processes for the plurality of time windows is defined as the sleeping state at the relevant detection time. In other words, when the desired time passes, the sleeping state at the desired time is judged based on the process in the time window in which the desired time is the end time.

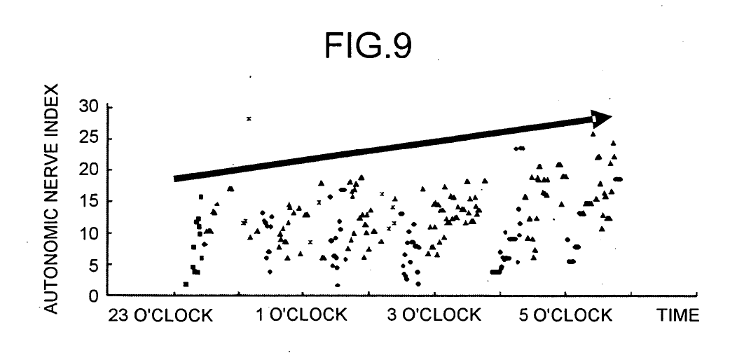

FIG. 9 is a chart showing the autonomic nerve index obtained during sleeping. REM sleeping or non-REM sleeping is judged from a magnitude relation between the autonomic nerve index and the threshold value. However, in an example shown in FIG. 9, the values of the autonomic nerve index corresponding to the REM sleeping and the non-REM sleeping, respectively are gradually increased as time passes. This is due to the effect by circadian rhythm. Thus, when the base itself of the autonomic nerve index is increased, judging the REM sleeping or the non-REM sleeping with a constant value used as the threshold value has a high possibility that a judgment error occurs.

Furthermore, the index of the autonomic nerves is affected by individual difference. Accordingly, in this case, if the values of the autonomic nerve index from 23 o'clock to 5 o'clock are compared with the same threshold value to judge the REM sleeping or the non-REM sleeping, the precise sleeping state cannot be judged.

Therefore, in the present embodiment, as explained above, for a sequence of sleeping state, the threshold value is set for every plurality of time windows including at least one cycle of sleeping and the sleeping state is judged based on the relevant threshold value. This excludes the effect by circadian rhythm and the like and enables the sleeping state to be judged more precisely.

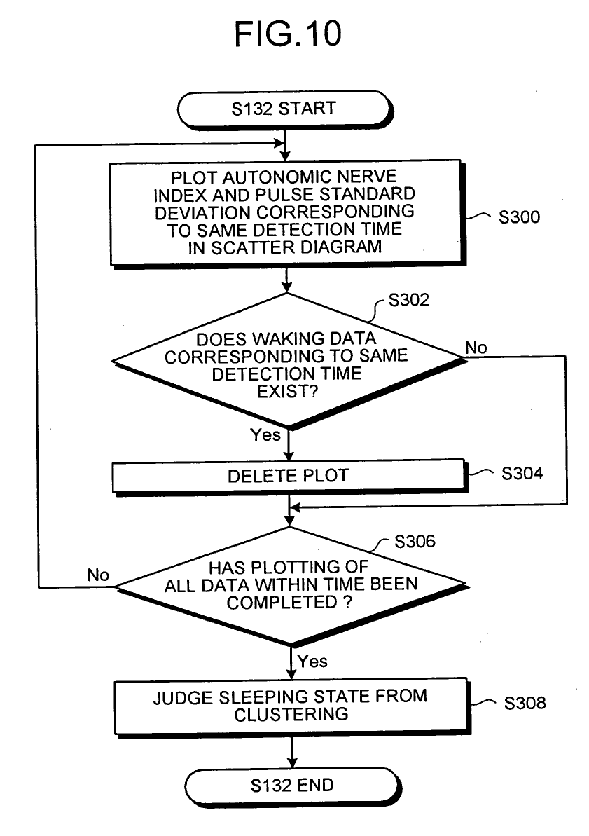

FIG. 10 is a detailed flowchart of the sleeping state judgment process (step S132) explained in FIG. 6 by the sleeping state judging unit 26. The sleeping state judging unit 26 according to the first embodiment may serve as a clustering unit and a sleeping state judging unit.

According to the first embodiment, since 100 minutes are set as the time window, the processing is performed for the data of 100 minutes. In other words, the processing is performed 100 times.

First, the autonomic nerve index data accumulated in association with an arbitrary detection time in step S120 and the pulse standard deviation accumulated in association with the same detection time in step S122 are plotted in a scatter diagram on plane coordinates (step S300). When the waking data accumulated in association with the same detection time as the detection time corresponding to this plot in step S110 exists (Yes in step S302), the corresponding plot is deleted from the scatter diagram (step S304). Thereby, the sleeping state can be judged only for the data during sleeping. Accordingly, the sleeping state can be judged more precisely.

The process from step S300 to step S304 is repeated until all the data within the time window is plotted in the scatter diagram (Yes in step S306). In the first embodiment, the process is repeated until 100 pieces of data are plotted in the scatter diagram. By clustering the plots in the scatter diagram, the sleeping state is judged (step S308).

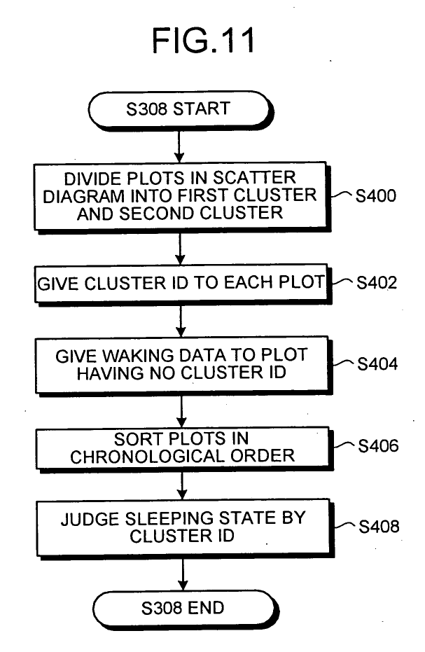

FIG. 11 is a detailed flowchart of the sleeping state judgment process by the clustering (step S308).

First, the scatter diagram is divided into two clusters using K-average algorithm (step S400). A cluster ID of the cluster whose center is closer to the origin is set as a first cluster. A cluster ID of the cluster whose center of the origin is farther from the origin is set as a second cluster.

The cluster ID is given to each piece of the data plotted in the scatter diagram (step S402). At this time, a cluster ID indicating that it is the waking data (step S404) is given to the data without the cluster ID. Next, 100 pieces of data each given the cluster ID are sorted in chronologic order (step S406).

Based on the cluster ID given to each piece of data sorted in chronologic order, the sleeping state is judged (step S408). Specifically, at the detection time corresponding to the data with the cluster ID of the first cluster, the subject is judged to be in the non-REM sleeping state. At the detection time corresponding to the data with the cluster ID of the second or other cluster, the subject is judged to be in the REM sleeping state.

The sleeping state judgment process according to the first embodiment may be realized by a sleeping state judging program stored in the storage unit 13, for example. The sleeping state judging program may be provided by being recorded on a computer-readable recording medium such as CD-ROM, a floppy™ disk (FD) and a digital versatile disk (DVD) in an installable form or an executable form of a file.

In this case, the sleeping state judging program is loaded on a main storage device by being read from the above-explained recording medium to be executed in the sleeping state judging device 100, and the respective units explained in the above software configuration are generated on the main storage device.

Furthermore, the sleeping state judging program according to the first embodiment may be configured to be provided by being stored on a computer connected to a network such as the Internet and being downloaded via the network.

While the present invention is explained according to the embodiment as shown above, various modifications or improvements can be made to the above-explained embodiment.

As a first modification, while in the present embodiment, the subject is judged to be in one of the state of REM sleeping and in the state of non-REM sleeping in the sleeping state judgment, the subject may be judged to be in one of three sleeping states, i.e., REM sleeping, deep non-REM sleeping, and shallow non-REM sleeping.

Specifically, the scatter diagram plots are divided into two clusters and K-average algorithm is applied to the data clustered as the non-REM sleeping, again. Specifically, for the data clustered as the non-REM sleeping, K-average clustering is performed using the autonomic nerve index data for both of the ordinate axis and the abscissa axis to plot on the plane coordinates. The plots below the center of gravity and near the origin are each given a cluster ID corresponding to the deep non-REM sleeping. Further, to each of the other plots, a cluster ID corresponding to the shallow non-REM sleeping is given.

By sorting the data with the cluster ID in chronologic order, the sleeping state at the respective detection times is judged to be one of the REM sleeping, shallow non-REM sleeping and deep non-REM sleeping.

The sleeping state to be judged is not limited to those employed in the first embodiment, and non-REM sleeping may be further subdivided to judge the levels of non-REM sleeping.

Furthermore, as a second modification, while in the first embodiment, the sleeping state is judged by clustering using K-average algorithm, as another example, a threshold value is set for each time window, and based on a magnitude relation with the relevant threshold value, the sleeping state may be judged. At this time, an average value of the data within the time window may be the threshold value. Furthermore, as another example, the threshold value may be determined based on the standard deviation of the data within the time window.

Thus, the data only needs to be divided into two groups for each time window, and the method is not limited to the present embodiment.

Furthermore, while the autonomic nerve index indicating the activity state of the autonomic nerves is calculated as the index reflecting the brain activities, which indicates the extent of changes in the activity state of the brain in the present embodiment, a cardiac beat period may be calculated instead as a third modification.

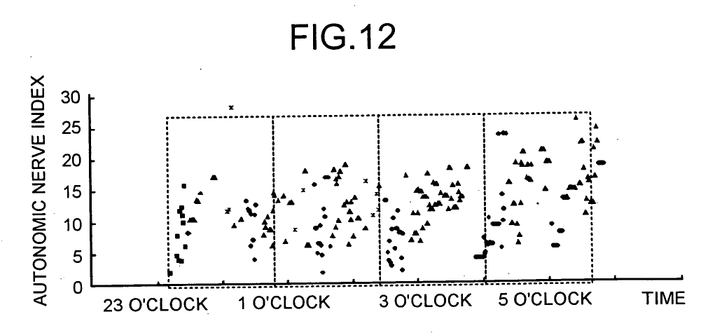

Next, the sleeping state judging device 200 according to a second embodiment will be described. FIG. 12 is a chart showing a time window set by the time window setting unit 27 of the sleeping state judging device 200 according to the second embodiment.

In the sleeping state judging device 100 according to the first embodiment, in consideration of real-time judgment, the time window is set at time intervals, each of which is shorter than the time interval of the time window. In the second embodiment, as shown in FIG. 12, the time window is set so that the end time of one time window is the start time of the next time window. In other words, in the second embodiment, at the start time included in a predetermined time window, the sleeping state is also judged from a judgment result of the data of a relevant time window.

Hence, the sleeping state judging device 200 according to the second embodiment can reduce the processing amount compared with the sleeping state judging device 100 according to the first embodiment.

The other configuration and processing of the sleeping state judging device 200 according to the second embodiment are similar to the configuration and processing of the sleeping state judging device 100 according to the first embodiment.

Additional advantages and modifications will readily occur to those skilled in the art. Therefore, the invention

1. A device for detecting a sleeping state of a subject, comprising:
a brain activity measuring unit that measures an index which reflects brain activities and which indicates an extent of changes in an activity state of a brain of the subject during sleeping at every given unit time; and
a sleeping state judging unit that judges the sleeping state at a given time in a time window which covers one cycle of sleeping, based on the index which reflects the brain activities and which is measured by the brain activity measuring unit at every given unit time within the time window.

2. The device according to claim 1, further comprising
a clustering unit that divides the indexes which reflect brain activities into two clusters based on the plurality of indexes which reflect the brain activities and which are measured at every given unit time,
wherein the sleeping state judging unit judges the sleeping state at the given time based on the clusters into which the indexes which reflect the brain activities and are measured at the given times in the time window are divided.

3. The device according to claim 1, wherein the brain activity measuring unit measures an activity state of an autonomic nerve of the subject as the index which reflects the brain activities.

4. The device according to claim 1, wherein the brain activity measuring unit measures a period of cardiac beat of the subject as the index which reflects the brain activities.

5. The device according to claim 1, wherein the sleeping state judging unit judges whether the subject is in a state of REM sleeping or in a state of non-REM sleeping based on the index which reflects the brain activities.

6. The device according to claim 1, further comprising
a sleeping and waking judging unit that judges whether the subject is in a waking state or in a sleeping state,
wherein the sleeping state judging unit judges the sleeping state, based on the index which reflects the brain activities and is measured by the brain activity measuring unit at the same time as a time when the sleeping and waking judging unit judges that the subject is in the sleeping state in the unit time.

7. The device according to claim 6, further comprising
a body movement detecting unit that detects a movement of a body of the subject,
wherein the sleeping state judging unit judges whether the subject is in the waking state or in the sleeping state, based on a result of detection by the body movement detecting unit.

8. The device according to claim 7, wherein the body movement detecting unit is an acceleration sensor which measures acceleration of the movement of the subject.

9. The device according to claim 7, wherein the body movement detecting unit is a pressure sensor that measures pressure generated by the movement of the subject.

10. The device according to claim 1, further comprising
a time window setting unit that sets plural time windows at time intervals shorter than the time window;
wherein the sleeping state judging unit judges the sleeping state at the given time, based on the index which reflects the brain activities and which is measured by the brain activity measuring unit in the time window including a latest time in the plural time windows when the given time is included in the plural time windows.

11. The device according to claim 1, further comprising
a time window setting unit that sets plural time windows at time intervals shorter than the time window; and
wherein the sleeping state judging unit judges the sleeping state at the given time, based on the index which reflects the brain activities and which is measured by the brain activity measuring unit at the every unit time in the time window in which the given time is latest time, when the given time elapses.

12. A method of detecting a sleeping state of a subject, comprising:
measuring an index which reflects brain activities and which indicates an extent of changes in an activity state of a brain of the subject during sleeping at every given unit time; and
judging the sleeping state at a given time in a time window which covers one cycle of sleeping, based on the index which reflects the brain activities and which is measured at every given unit time within the time window, among the measured indexes which reflects the brain activities.

13. A computer program product having a computer readable medium including programmed instructions for performing a sleeping state detecting process according to which a sleeping state of a subject is judged, wherein the instructions, when executed by a computer, cause the computer to perform:
measuring an index which reflects brain activities and which indicates an extent of changes in an activity state of a brain of the subject during sleeping at every given unit time; and
judging the sleeping state at a given time in a time window which covers one cycle of sleeping, based on the index which reflects the brain activities and which is measured at every given unit time within the time window, among the measured indexes which reflects the brain activities.
